(19) United States
(12) Patent Application Publication
(10) Pub. No.: US 2005/0158365 A1
(43) Pub. Date: Jul. 21, 2005

(54) Drug delivery device with mechanical locking mechanism

(76) Inventors: David Watson, San Jose, CA (US); George Purtell, Westford, MA (US); Brian Levy, Rochester, NY (US); Dominic V. Ruscio, Webster, NY (US); Jay F. Kunzler, Canandaigua, NY (US); Michael M. Schmidt, Cambridge, MA (US); Matthew S. Jonasse, Sodus, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 11/013,230

(22) Filed: Dec. 15, 2004

Related U.S. Application Data
(60) Provisional application No. 60/531,879, filed on Dec. 22, 2003.

(51) Int. Cl. A61K 9/22; A61F 2/00

(52) U.S. Cl. 424/427; 604/890.1

(57) ABSTRACT

A drug delivery device for placement in the eye includes a drug core comprising a pharmaceutically active agent, and a holder that holds the drug core. The holder is made of a material impermeable to passage of the active agent and includes at least one opening for passage of the pharmaceutically agent therethrough to eye tissue. The holder is mechanically secured to a suture tab.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes at least one opening for passage of the pharmaceutically active agent therethrough to eye tissue. Particularly, the device of this invention is secured to a suture tab by a mechanical locking mechanism.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include a suture tab for securing the device to a structure of, for example, the eye. When the device contains an integral suture tab, a compromise may be reached between the properties necessary to provide a suitable holding device for the drug core and the properties necessary for a suitable suture tab. Therefore, to optimize the properties of the materials used, it may be desirable to form the drug holder and suture tab from different materials. When the suture tab is made of a material different than the holder material, it must be secured to the holder so that the suture tab and holder do not separate while in use.

A conventional method for joining the suture tab to the holder is the use of room temperature vulcanizable (RTV) adhesive. Although entirely satisfactory in many applications, the use of RTV adhesive can add additional steps to the process of making drug delivery devices and may allow the suture tab and holder to separate during use. Therefore, a new method for securing the holder to the suture tab is needed.

### BRIEF DESCRIPTION OF THE DRAWINGS

FIG. 1 is a cross-sectional view of a first embodiment of a drug delivery device of this invention.

FIG. 2 is an exploded perspective view of the drug delivery device of FIG. 1.

FIG. 3 is an exploded perspective view of a second embodiment of a drug delivery device of this invention.

FIGS. 4a and 4b are cross-sectional views of a third embodiment of a drug delivery device.

FIG. 5 is an exploded perspective view of the drug delivery device of FIG. 4b.

### SUMMARY OF THE INVENTION

According to a first embodiment, this invention relates to a drug delivery device for placement in the eye, comprising a drug core comprising a pharmaceutically active agent; a holder that holds the drug core, the holder being made of a material impermeable to passage of the active agent and including an opening for passage of the pharmaceutically active agent therethrough to eye tissue, a suture tab having a suture hole at an end thereof, the other end containing a hole for receiving the holder; and a mechanical locking mechanism for securing the holder to the suture tab. The mechanical locking mechanism can be any suitable means such as a grommet type device or tapered tabs.

This invention further relates to an assembly for containing the device for packaging and shipping. In one embodiment the assembly may comprise a package for storing an implantable medical device during storage and shipping, comprising an upper surface, a first flange extending upwardly from the upper surface and defining a containment region for containing the device, said containment region including a support surface for supporting the device in the containment region; a second flange extending upwardly from the upper surface, said second flange surrounding the first flange and including an upper flange surface for sealing of lidstock thereto; and at least one side wall extending downwardly from the upper surface and serving to support the package on a work surface, further comprising a recess extending below the device support surface in the containment region, wherein the first flange comprises two protrusions extending upwardly from the upper surface and defining the containment region, and the recess has the form of an elongated groove separating the two protrusions and extending transversely to the containment region, wherein the two protrusions are arcuate, wherein the maximum width between inner surfaces of an individual protrusion is 10 mm.

### DETAILED DESCRIPTION OF PREFERRED EMBODIMENTS

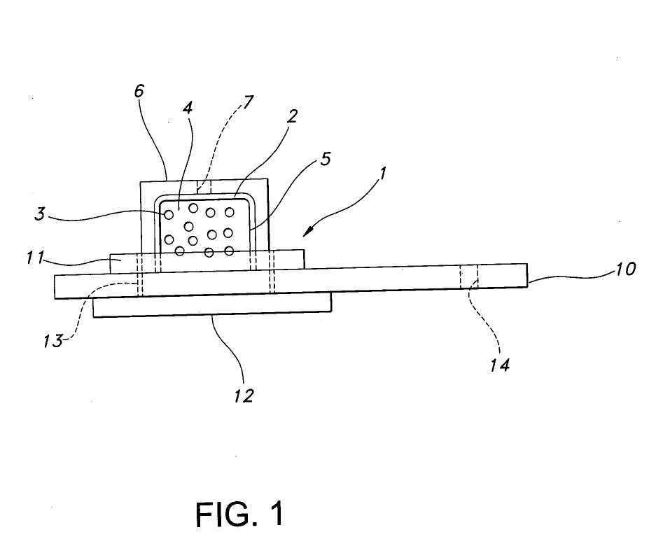

FIG. 1 illustrates a first embodiment of a device of this invention. Device 1 is a sustained release drug delivery device for implanting in the eye. Device 1 includes inner drug core 2 including a pharmaceutically active agent 3.

This active agent 3 may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecarnil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impeding agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrize inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal antiinflammatory agents such as ibuprofen; nutrients and vitamins; enzyme inhibitors; antioxidants; anticataract drugs; aldose reductase inhibitors; cytoprotectants; cytokines, cytokine inhibitors, and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent 3 employed is fluocininolone acetonide.

As shown in the illustrated embodiment, active agent 3 may be mixed with a matrix material 4. Preferably, matrix material 4 is a polymeric material that is compatible with body fluids and the eye. Additionally, matrix material 4 should be permeable to passage of the active agent 3 therethrough, particularly when the device is exposed to body fluids. For the illustrated embodiment, the matrix material 4 is polyvinyl alcohol (PVA). Also, in this embodiment, inner drug core 2 may be coated with a coating 5 of additional matrix material, which may be the same or different from material 4 mixed with the active agent 3. For the illustrated embodiment, the coating 5 employed is also PVA.

Materials suitable as coating 5 would include materials that are non-bioerodible and are permeable or semi-permeable to the active agent. Preferably, the coating material will be release rate limiting. Suitable polymers, depending upon the specific active agent, would include polyvinyl alcohol, ethylene vinyl acetate, polylactic acid, nylon, polypropylene, polycarbonate, cellulose, cellulose acetate, polyglycolic acid, polylactic glycolic acid, cellulose esters or polyether sulfone. Coating 5 may also be any of the various semipermeable membrane-forming compositions or polymers such as those described in US Patent Publication No. 2002/0197316 (hereby incorporated by reference). Coating 5 may also include plasticizer and pharmaceutically acceptable surfactant such as those described in US Patent Publication No. 2002/0197316.

Further examples of semipermeable polymers that may be useful according to the invention herein can be found in U.S. Pat. No. 4,285,987 (hereby incorporated by reference), as well as the selectively permeable polymers formed by the coprecipitation of a polycation and a polyanion as described in U.S. Pat. Nos. 3,541,005; 3,541,006 and 3,546,142 (hereby incorporated by reference.

Device 1 includes a holder 6 for the inner drug core 2. Holder 6 is made of a material that is impermeable to passage of the active agent 3 therethrough. Since holder 6 is made of the impermeable material, a passageway 7 is formed in holder 6 to permit active agent 3 to pass therethrough and contact eye tissue. In other words, active agent 3 passes through any permeable matrix material 4 and permeable or semi-permeable coating 5, and exits the device through passageway 7. Holder 6 is continuous with a base portion 12. The base portion 12 is sized to be larger than the opening 13 contained in an end opposite a suture hole 14 in suture tab 10. Base 12 allows the holder 6 to be inserted through opening 13 configured in suture tab 10 to receive holder 6 yet preventing the holder 6 from passing completely through opening 13 of suture tab 10. For the illustrated embodiment, the holder 6 is made of silicone, especially polydimethylsiloxane (PDMS) material.

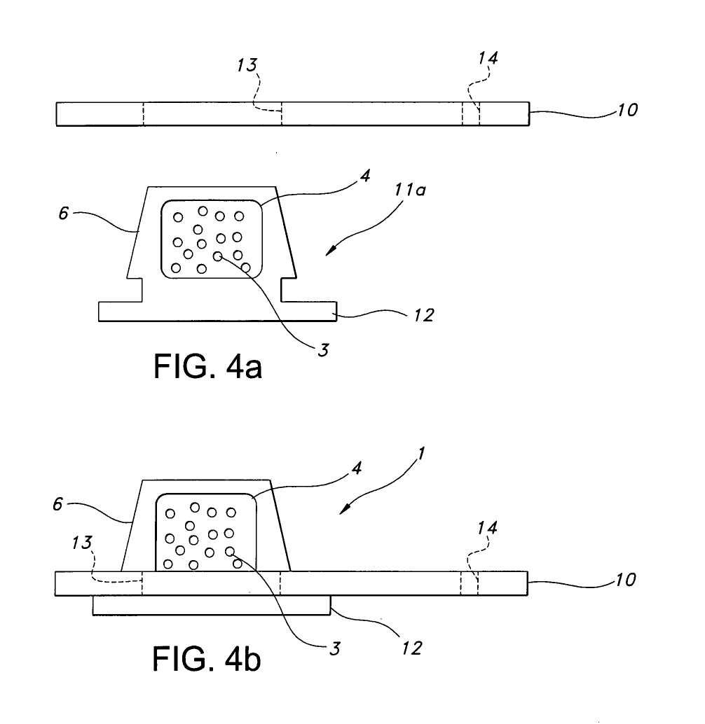

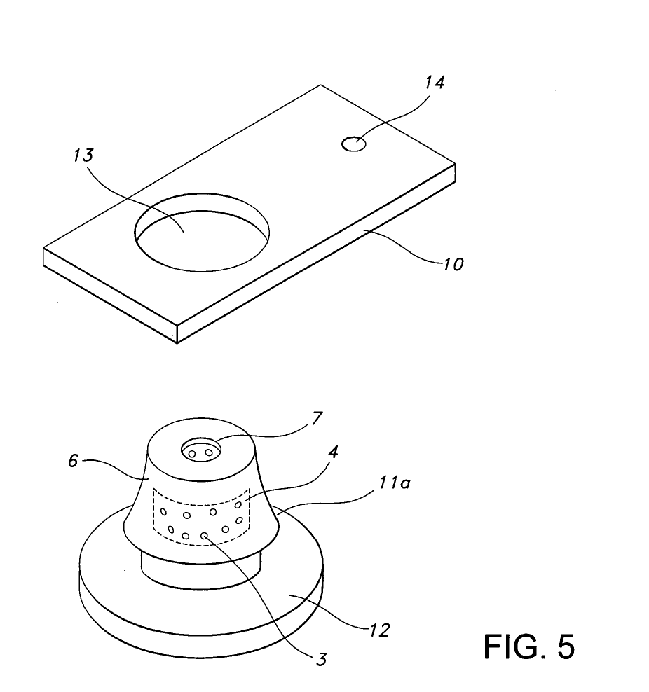

The illustrated embodiment includes a tab 10 which may be made of a wide variety of materials, including those mentioned above for the matrix material and/or the holder. Tab 10 may be provided in order to attach the device to a desired location in the eye, for example, by suturing. For the illustrated embodiment, tab 10 is made of PVA and is adhered to holder 6 with mechanical locking means 11. Mechanical locking means 11 may be a grommet 11, a tapered tab 11a (shown in FIGS. 4a, 4b and 5), or the like.

A wide variety of materials may be used to construct the device 1 of the present invention. The only requirements are that they are inert, non-immunogenic and of the desired permeability. Materials that may be suitable for fabricating the device 1 include naturally occurring or synthetic materials that are biologically compatible with body fluids and body tissues, and essentially insoluble in the body fluids with which the material will come in contact.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

According to preferred embodiments, the holder 6 is also extracted to remove residual materials therefrom. For example, in the case of silicone, the holder 6 may include lower molecular weight materials such as unreacted monomeric material and oligomers. It is believed that the presence of such residual materials may also deleteriously affect adherence of the holder surfaces. The holder 6 may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder 6 may be plasma treated, following extraction, in order to increase the wettability of the holder 6 and improve adherence of the drug core 4 to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like or appropriate mixtures thereof including inert gases such as argon. Examples of mixed media include oxygen/argon or hydrogen/methanol. Typically, the plasma treatment is conducted in a closed chamber at an electric discharge frequency of 13.56 MHz, preferably between about 20 to 500 watts at a pressure of about 0.1 to 1.0 torr, preferably for about 10 seconds to about 10 minutes or more, more preferably about 1 to 10 minutes.

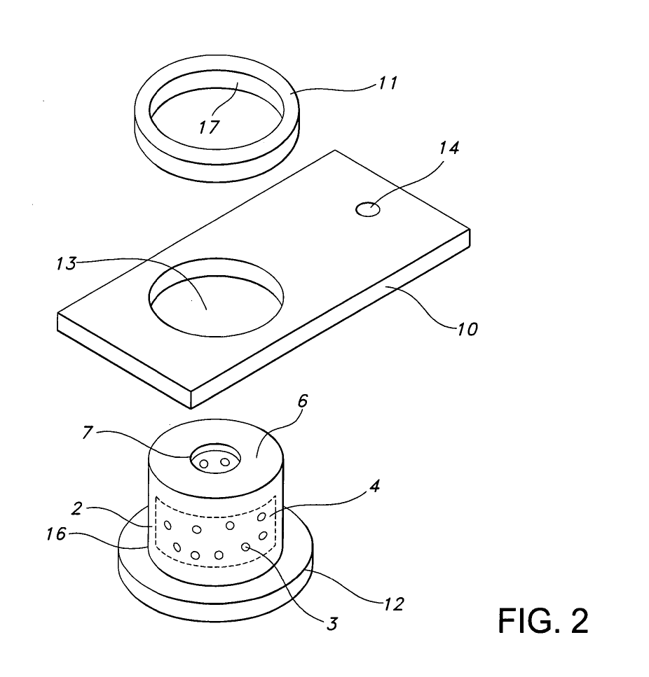

FIG. 2 is an exploded view of the device of FIG. 1 showing how the device 1 may be assembled. For the illustrated embodiment, the active agent 3 may be provided in the form of a micronized powder, and then mixed with an aqueous solution of the matrix material 4, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

An embodiment of the invention herein may be prepared in the following manner. A tube of the polymeric material is sliced cross-wise to create about 0.5 mm long segments of the tube (grommet). One grommet 11 is necessary for each final assembly.

PVA is cast and cured and then cut into strips about 48 mm wide. The cured PVA strip is formed into a suture tab 10 using procedures well known to those skilled in the polymeric arts, e.g., punch press. Because the size of PVA film can be dependent upon hydration levels, the cured PVA strip should be formed into the suture tab a short time after the PVA strip is cut from the cast and cured PVA film.

A small amount of PVA solution is placed into each previously extracted array cup and the prepared core 2 containing the active 3 is inserted. The core 2 and the PVA solution are then cured.

The backing sheet is placed onto the array containing the tablet so that the ends of the backing sheet do not extend more than about 1.5 mm past the ends of the array and that each cup is covered by the backing sheet. The backing sheet is then adhered to the array, for example by adhesive under vacuum. The array and backing sheet are allowed to dry for a sufficient amount of time (e.g., 24 hours).

The array with cured tablets is then cut into individual sample cups with attached backing sheet (for example by stamping). The attached backing sheet forms a flange. The holder 6 is held in place (for example, under vacuum) on an assembly plate and a suture tab 10 is placed over each holder 6 and gently maneuvered onto the holder 6 until the suture tab 10 is flush against the base portion 12. A fine bead of adhesive, for example RTV adhesive, is then placed around the top perimeter 16 of the holder 6. A grommet 11 is then advanced over the cup so that the adhesive wets the inner surface 17 of the grommet 11. The grommet 11 is advanced onto the holder 6 until the grommet 11 holds the suture tab 10 flush to the base portion 12 of the cup. This process is then repeated for each holder 6.

The adhesive is then allowed to dry.

The assembled device is then shaped to its final dimensions, inspected and packaged for use or storage.

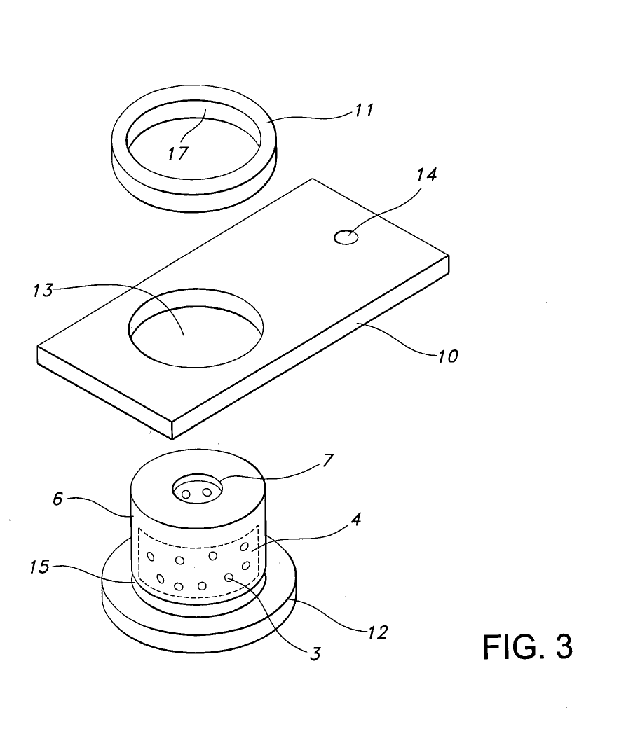

FIG. 3 illustrates another embodiment. In this embodiment, holder 6 contains a grooved or recessed portion 15 adjacent to base 12. The grooved portion 15 receives the grommet 11 for a mechanically engaged locking means.

FIGS. 4a and 4b are cross-sectional views of a third embodiment of the drug delivery device. In this embodiment, holder 6 comprises a tapered tab 11a that engages with suture tab 10 through a friction fit with opening 13.

FIG. 5 is an exploded perspective view of one embodiment of the device of FIG. 4b. In this embodiment, the tapered tab 11a is circumferential around holder 6. It should be understood that other configurations (not shown) of the tapered tab are envisioned such as two laterally placed tabs, four-square arrangements of tabs, etc. In fact, any number tapered tabs may be used to secure the holder to the suture tab.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is generally intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

It should be understood that the preferred device comprises a suture tab. However, a suture tab is not necessary for therapeutic operation of the device.

The device is typically provided to the end user in a sealed sterilized package, for example by gamma irradiation, for example, such as is disclosed in U.S. application Ser. No. 10/183,804, the contents of which are incorporated by reference herein.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

1. A drug delivery device for placement in the eye, comprising:
a drug core comprising a pharmaceutically active agent;
a holder that holds the drug core, the holder being made of a material impermeable to passage of the active agent and including an opening for passage of the pharmaceutically agent therethrough to eye tissue, the holder having a base;
a suture tab having a suture hole at an end thereof, an end opposite the suture hole having an opening for receiving the holder;
and a mechanical mechanism for retaining the holder in the suture tab.

2. The device of claim 1, wherein the impermeable material comprises silicone.

3. The device of claim 1, wherein the mechanical mechanism for retaining the holder in the suture tab is a grommet.

4. The device of claim 1, wherein the mechanical mechanism for retaining the holder in the suture tab is a tapered tab.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder comprises a cylinder that surrounds the drug core, and an end of the cylinder includes the opening.

8. The device of claim 1, wherein the drug core is cylindrical.

9. The device of claim 1, wherein the drug core is coated with a material permeable to said active agent.

10. The device of claim 1, comprising a mixture of pharmaceutically active agents.

11. A package for storing an implantable medical device during storage and shipping, comprising:
an upper surface;
a first flange extending upwardly from the upper surface and defining a containment region for containing the device, said containment region including a support surface for supporting the device in the containment region;
a second flange extending upwardly from the upper surface, said second flange surrounding the first flange and including an upper flange surface for sealing of lidstock thereto; and
at least one side wall extending downwardly from the upper surface and serving to support the package on a work surface,
further comprising a recess extending below the device support surface in the containment region,
wherein the first flange comprises two protrusions extending upwardly from the upper surface and defining the containment region, and the recess has the form of an elongated groove separating the two protrusions and extending transversely to the containment region,
wherein the two protrusions are arcuate, wherein the maximum width between inner surfaces of an individual protrusion is 10 mm, and
wherein the implantable medical device is the device of claim 1.

12. The package of claim 11 wherein the implantable medical device is the device of claim 2.

13. The package of claim 11 wherein the implantable medical device is the device of claim 3.

14. The package of claim 11 wherein the implantable medical device is the device of claim 4.

15. An assembly comprising:
(a) a medical device implantable in the human eye;
(b) a package for storing the device during storage and shipping;
wherein the medical device is the device of claim 1.

16. The assembly of claim 15 wherein the medical device is the device of claim 2.

17. The assembly of claim 15 wherein the medical device is the device of claim 3.

18. The assembly of claim 15 wherein the medical device is the device of claim 4

19. The assembly of claim 15 wherein the assembly is sterilized.

20. The assembly of claim 15 wherein the assembly is sterilize by gamma irradiation.

21. The assembly of claim 16 wherein the assembly is sterilized.

22. The assembly of claim 16 wherein the assembly is sterilized by gamma irradiation.

23. The assembly of claim 17 wherein the assembly is sterilized.

24. The assembly of claim 17 wherein the assembly is sterilized by gamma irradiation.

25. The assembly of claim 18 wherein the assembly is sterilized.

26. The assembly of claim 18 wherein the assembly is sterilized by gamma irradiation.
